(19) United States
(12) Patent Application Publication
(10) Pub. No.: US 2015/0238279 A1
(43) Pub. Date: Aug. 27, 2015

(54) DISCLOSING LIGHT OF DENTAL RESTORATIVE MATERIALS FOR A HIGH SPEED HANDPIECE SETTING AND DISCLOSING LIGHT DEVICE OF DENTAL RESTORATIVE MATERIALS

(71) Applicant: GNATUS EQUIPAMENTOS MÉDICO ODONTOLÓGICOS LTDA, Ribeirao Preto (BR)

(72) Inventor: Valter Flavio Scalco, Londrina (BR)

(73) Assignee: GNATUS EQUIPAMENTOS MÉDICO ODONTOLÓGICOS LTDA, Ribeirao Preto (BR)

(21) Appl. No.: 14/191,632

(22) Filed: Feb. 27, 2014

(51) Int. Cl. A61C 1/08; A61B 1/06; A61C 1/00; A61B 1/24

(52) U.S. Cl. CPC A61C 1/088; A61B 1/24; A61B 1/0684; A61B 1/0638; A61C 1/0061

(57) ABSTRACT

Disclosing light of dental restorative materials for a high speed handpiece setting and disclosing light device of dental restorative materials, comprising a light-emitting (1) based on the emission of ultraviolet light (2) through LED (3), capable of being installed on a high speed handpiece setting (4) associated to white light LED (3B), or a dedicated light-emitting device (5), which, when focusing the light beam on the tooth, evidences, through difference in fluorescence, restorative materials (6) from the original tooth structure (7).

### FIELD OF THE INVENTION

This invention relates to an unprecedented disclosing light of dental restorative materials for a high speed handpiece setting and disclosing light device of dental restorative materials, based on an LED emitting ultraviolet light with the ability to highlight the presence of different types of dental restorative materials, making possible its visualization, distinction and removal in a selective manner, without further prejudice to the genuine tooth structure.

The invention's field of application is the restorative dentistry and can be installed in a high speed handpiece setting as well in a specific light-emitting device.

### BACKGROUND OF THE INVENTION

It is known by a skilled person in the art that the biggest development recently recorded in the history of dentistry, which has caused radical changes in the restoration of tooth structures way, was the advent of dental adhesives.

From 1955 and onwards, the first reports emerged about the possibility of the accession of restorative materials to a tooth structure throughout the researcher Michael Buonocore (Carvalho et al., 2004). This new discovery has provided a wide field of research, with the possibility of conducting adhesive restorations with minimally invasive preparations, or even without any preparation.

In this context, the major purpose of the known adhesive restorative materials is to preserve the tooth structure, and also to aesthetically achieve restoration of color and texture, as closely mimicked, by reaching the optimum point of practically being impossible to differentiate such materials from natural teeth tissues.

Furthermore, as any restorative material has its pre-stipulated life cycle, and thus, should be replaced one further day; and according to the discussion above it may not be possible to distinguish them, it is most likely that the healthy tooth structure may be unnecessarily worn, against all conservative philosophy and best practices in the area.

Therefore, it is usual to have not only the removal of healthy tissue but also, in the same harmful manner, the insufficient removal of adhesive restorative material, which results in poor cleaning of the tooth under treatment.

In this scenario, the visual acuity and professional experience is determinant to obtain a satisfactory result regarding the removal of the restoration, which primarily requires time, skill and proper equipment.

Studies have proven that the light source represented by the solidary reflector to the giving device directly interferes on the fidelity of perceptible shades by the human eyes, this being a limitation which, added to the visual perfection achieved in restorative materials, affects the final quality of the removal procedure.

Viewed from this angle, it can be stated that professionals in this field must have assistance by means of facilitating the visualization and distinction between dental restorative materials and tissue adhesives, by the factors that have already been widely commented.

The current prior art does not anticipate any specific device that distinguishes restorative materials when placed on the teeth; however, seeking to settle this issue, the PI 0312034-1 “Composite Resin, Artificially Coloured, for Base of Restoration in Posterior Teeth and/or lower teeth” requires a resin with a different colour of known composites and also different from the colour of the teeth that may be the basis, getting in contact with the tooth, thereby avoiding the wear of the healthy dental tissue, once the different basal colouring will determine the limit of wear and removal the composite resin.

The solution above-mentioned has an inventive concept, a new formulation of resin with a different color from the already known composites, i.e., in the material used and not in an independent appeal that makes the disclosing of the resin, and in the case of this application is not necessary to alter the composition in order to stand out in relation to dental tissue.

### SUMMARY OF THE INVENTION

Considering a way to settle the drawbacks and technical limitations commented above, the inventor, a person with considerable knowledge in the field, after deep studies and research, has created a disclosing light of dental restorative materials for a high speed handpiece setting and disclosing light device of dental restorative materials; the invention consists of a LED emitting ultraviolet light (UV), which along with auxiliary components necessary to the operation, embed a high speed handpiece setting or even configure a light-emitting device, with the purpose of, through difference of the fluorescence when the emitting of UV light at a certain wavelength range, evidencing the restorative materials from the original tooth structure.

The disclosing light of dental restorative materials for a high speed handpiece setting and disclosing light device of dental restorative materials, when embedded in high speed handpiece setting, and with a white light LED, takes place inside a gauntlet with a rear adapter for the passage of compressed air responsible for the rotation of the rotor, for the fluid to the cooling spray of the tip of the high rotation pin, and for the transpassing of the contact wiring of LED UV and white light LED with external power source.

In the disclosing light of dental restorative materials for a high speed handpiece setting and disclosing light device of dental restorative materials, when embedded in high speed handpiece setting, a switch is installed between the double pneumatic valve of the equipment and the reversing switch of polarity of the high speed handpiece setting, which upon in touch with air from said valve, and in the proper polarity, will activate distinctly the LED UV or the white light LED.

In the disclosing light of dental restorative materials for a high speed handpiece setting and disclosing light device of dental restorative materials, when embedded in high speed handpiece setting, LED emitting UV light and white light UV are positioned on the front lower surface of the gauntlet of the high speed handpiece setting, so that, when the high speed handpiece setting works, the user, by reversing switch of polarity, can choose the disclosing light with LED UV, or the simples lightning of work the area with white light LED, without any prejudice to the operation of said setting and its spray system.

In the disclosing light of dental restorative materials for a high speed handpiece setting and disclosing light device of dental restorative materials, when embedded in high speed handpiece setting, LED emits UV light at a wavelength range between 320 and 400 nm+/−5%.

The disclosing light of dental restorative materials for a high speed handpiece setting and disclosing light device of dental restorative materials, when configuring a light-emitting device, has a bipartite structure, which houses the LED emitting UV light and its complements.

The disclosing light of dental restorative materials for a high speed handpiece setting and disclosing light device of dental restorative materials, when configuring a light-emitting device, is powered by an external power source, where the charging of internal battery takes place in suitable base.

The disclosing light of dental restorative materials for a high speed handpiece setting and disclosing light device of dental restorative materials, when configuring a light-emitting device, has an eye protector and a conductive tip of UV light.

The disclosing light of dental restorative materials for a high speed handpiece setting and disclosing light device of dental restorative materials, when configuring a light-emitting device, has a specific electronic board for LED and a lens.

The disclosing light of dental restorative materials for a high speed handpiece setting and disclosing light device of dental restorative materials, when configuring a light-emitting device, is operated by buttons strategically placed in its structure.

In the disclosing light of dental restorative materials for a high speed handpiece setting and disclosing light device of dental restorative materials, when configuring a light-emitting device, LED emits UV light in the wavelength range between 320 and 400 nm+/−5%.

The disclosing light of dental restorative materials for a high speed handpiece setting and disclosing light device of dental restorative materials, as much applied to a high speed handpiece setting, as configuring a device, besides showing the restorative materials and its selective removal also provides better visualization of caries and cracks in the tooth structure, aiding the diagnosis.

### BRIEF DESCRIPTION OF DRAWINGS

The following explains the invention with reference to the attached drawings in which are represented in an illustrative and non-limiting way:

FIG. 1: Perspective view of the disclosing light of dental restorative materials for high speed handpiece setting;

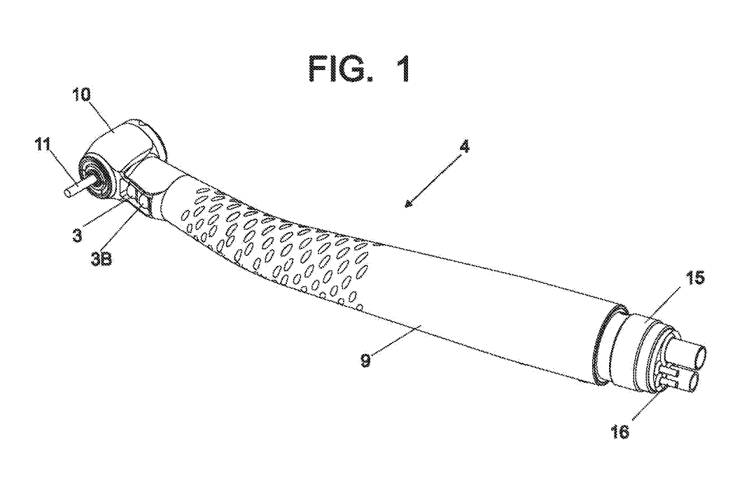

FIG. 2: Exploded perspective view showing main components of the disclosing light of dental restorative materials for a high speed handpiece setting;

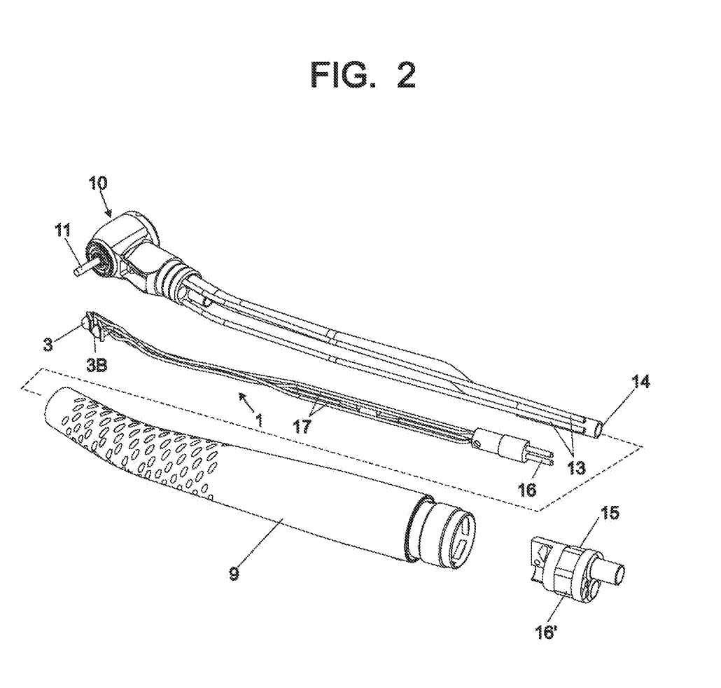

FIG. 3: Sectional view of the disclosing light of dental restorative materials for high speed handpiece setting;

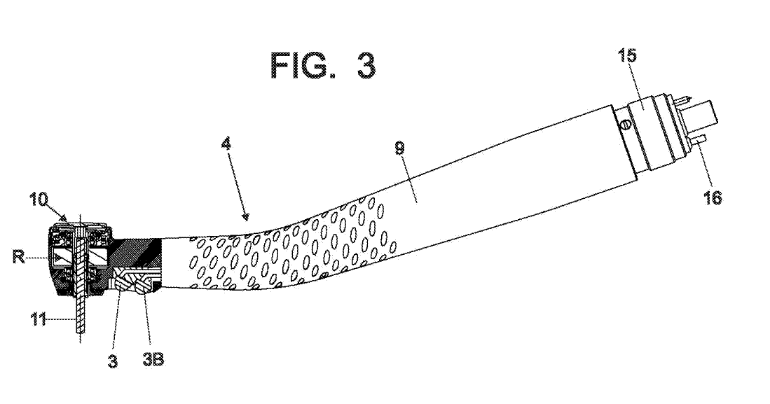

FIG. 4: Schematic view of the infrastructure necessary to the operation of the disclosing light of dental restorative materials for a high speed handpiece setting;

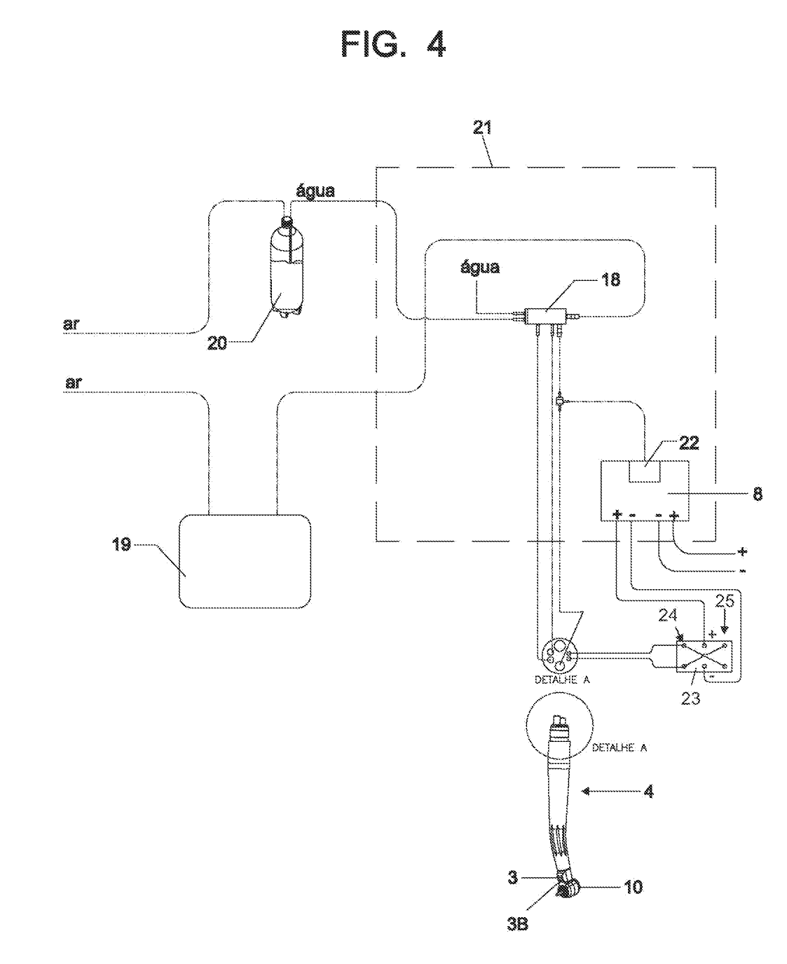

FIG. 5: Perspective view of the disclosing light device of dental restorative materials;

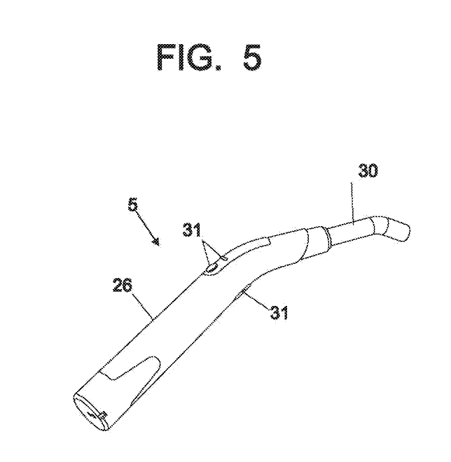

FIG. 6: Exploded perspective view showing the main components of the disclosing light device of dental restorative materials;

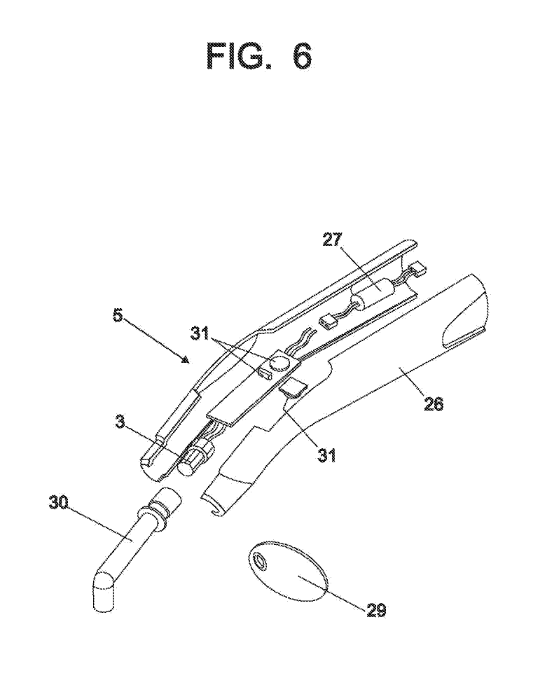

FIG. 7: Sectional view of the disclosing light device of dental restorative materials;

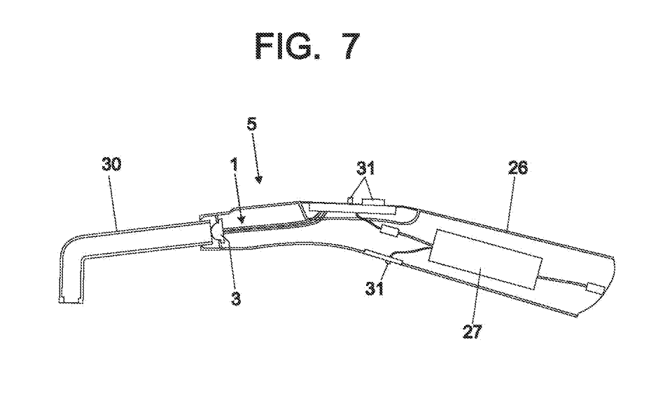

FIG. 8: Schematic view of the infrastructure necessary to the operation of the disclosing light device of dental restorative materials;

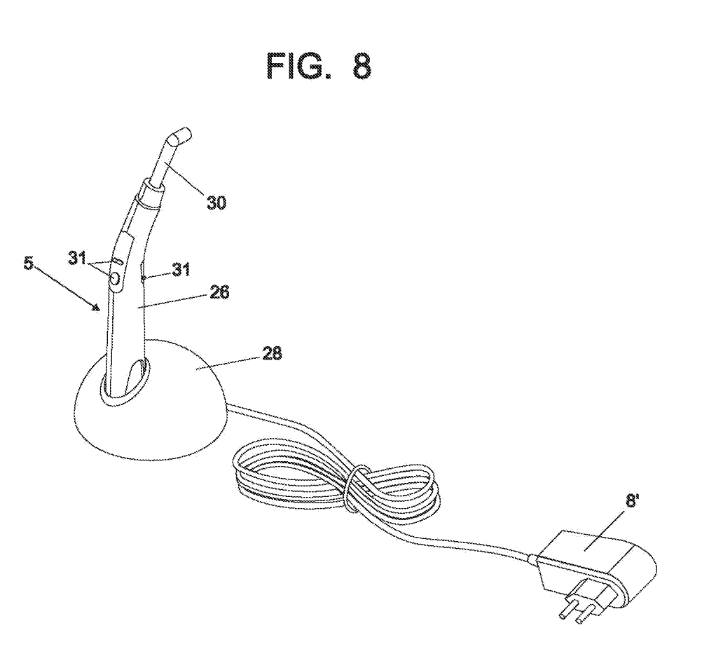

FIG. 9: Perspective view showing the use of the disclosing light of dental restorative materials for a high speed handpiece setting with LED UV;

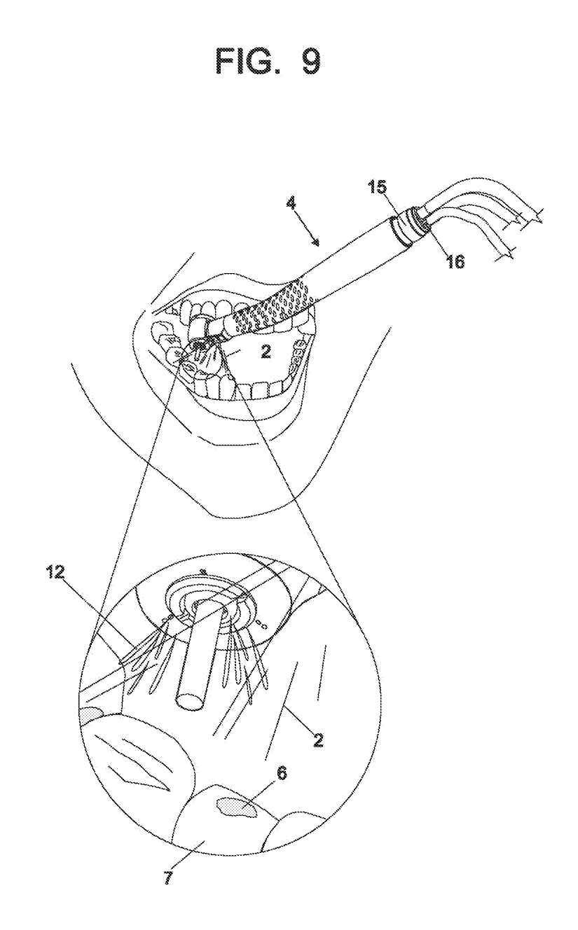

FIG. 10: Perspective view showing the use of the disclosing light of dental restorative materials for a high speed handpiece setting with white light LED;

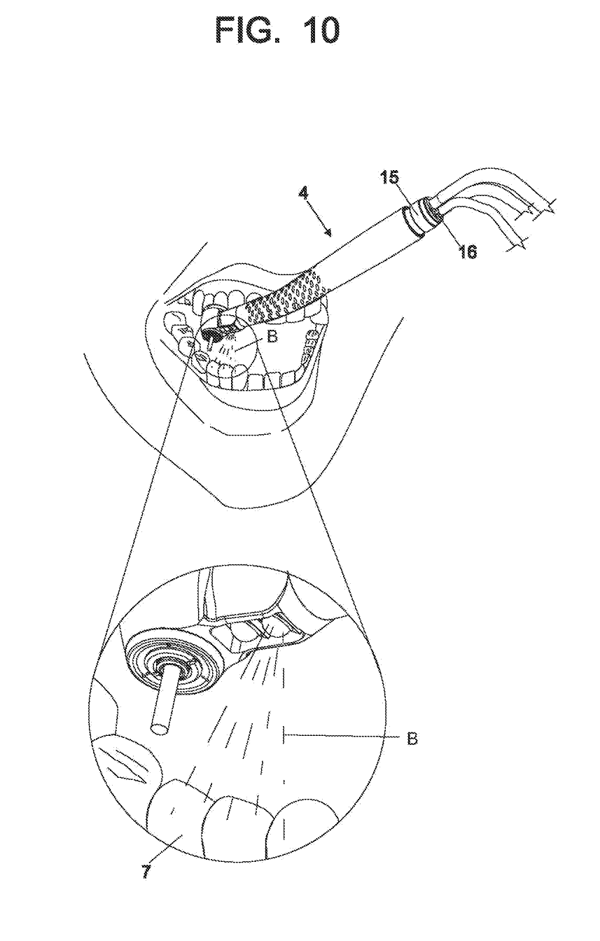

FIG. 11: Perspective view showing the use of the disclosing light device of dental restorative materials.

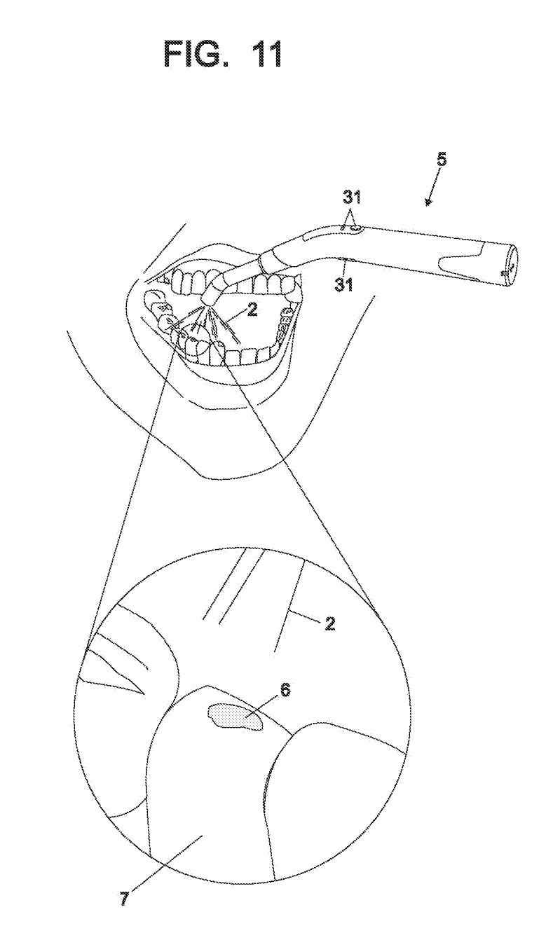

### DETAILED DESCRIPTION OF THE PREFERRED EMBODIMENT

The disclosing light of dental restorative materials for a high speed handpiece setting and disclosing light device of dental restorative materials, object of this invention consists of a light-emitting (1) based on the emission of ultraviolet light (2) through LED (3), capable of being installed on a high speed handpiece setting (4) associated to white light LED (3B) or a dedicated light-emitting device (5), which, when focusing the light beam on the tooth, evidences, through difference in fluorescence, restorative materials (6) from the original tooth structure (7).

More specifically, this light-emitting (1) is based on a LED (3) capable of emitting ultraviolet light (2) generated by a power source (8), said light-emitting (1) can be applied to high speed handpiece setting (4) associated to white light (B) LED (3B) or dedicated UV light-emitting device (5), both in a wavelength range from 320 to 400 nm+/−5%. When installed on high speed handpiece setting (4), the light-emitting (1) takes place inside a gauntlet (9) with LED (3) and LED (3B) positioned on its front lower surface, adjacent to the head (10), which receives the rotor (R), high rotation pin (11) and cooling water spray (12) from the respective tubes (13, 14) of water and air. Meanwhile, in the rear area of the gauntlet (9), an adapter (15) is provided with holes for the passage of such tubes (13, 14), and holes (16′) for the transpassing of contacts (16) of the LED (3) and LED (3B) wiring (17) toward the power source (8).

Operationally, the LED (3) lighting occurs with the pneumatic valve (18) opening, which receives air from the compressor, which also supplies the footswitch (19) of the high speed handpiece setting, the water tank (20) and the tub of the equipment (21). The airflow pushes an air-electronic switch (22), which signals the release of energy to the reversing switch (23), which, in the position (24), lights the LED (3), simultaneously to the activation of the rotor and the water spray, thereby releasing the UV light beam (2) toward to the tooth, said UV light beam, as already mentioned, through difference in fluorescence, distinguishes restorative materials (6) from the original tooth structure (7). On the other hand, the LED (3B) lightning also occurs through the pneumatic valve (18) opening, which pushes an air-electronic switch (22), signalizing for the release of energy to the reversing switch (23); which, in the position (25), lights the LED (3B) simultaneously to the activation of the rotor and the water spray; said LED (3B) emits the white light beam (B) towards the tooth, very useful at work situations where the dental reflector doesn't provide suitable lighting. If the reversing switch (23) is not in any of above mentioned conditions, none of the LED's (3 and 3B) is activated, and the equipment works as a high rotation pen.

Keeping the same inventive concept, but without LED (3B), the light-emitting (1) may be applied to dedicated UV (2) light-emitting device (5) in the wavelength range between 320 and 400 nm +/−5% with the same disclosing purpose, which is composed by a bipartite structure (26) containing the LED (3) with lens and related circuit, whose battery (27) is powered by external power source (8′) connected to the base (28) suitable for receiving said device (5), which also has an eye protector (29) and a tip (30) conductive of

UV light. Finally, the operation of the device (5) is made by buttons (31) strategically positioned in its structure (26).

What claimed is:

1. Disclosing light of dental restorative materials for a high speed handpiece setting and disclosing light device of dental restorative materials, wherein it is based on a LED (3) capable of emitting ultraviolet light (2) and a white light LED (3B) generated by a power source (8), said light-emitting can be applied to high speed handpiece setting (4) or UV light-emitting device (5), both UV in a wavelength range from 320 to 400 nm+/−5%.

2. Disclosing light of dental restorative materials for a high speed handpiece setting and disclosing light device of dental restorative materials, according to claim 1, wherein in the high speed handpiece setting (4), the light-emitting (1) taking place inside the gauntlet (9) with the LED (3) and LED (3B) located on its front lower surface, adjacent to the head (10); in the rear area of the gauntlet (9) an adapter (15) is provided with holes for the passage of such tubes (13, 14), and holes (16′) for the transpassing of contacts (16) of the LED (3) and LED (3B) wiring (17) toward the power source (8), the LED (3) lighting occurs through the pneumatic valve (18) opening, whose air pushes an air-electronic switch (22), which signalizes for the release of energy to the reversing switch (23); in the position (24), it lights the LED (3); in the position (25), it lights the LED (3B). 3) Disclosing light of dental restorative materials for a high speed handpiece setting and disclosing light device of dental restorative materials, according to claim 1, wherein the light-emitting applied to dedicated UV light-emitting device (5) in the wavelength range between 320 and 400 nm+/−5% is composed by a bipartite structure (26) containing the LED (3) with lens and related circuit, whose battery (27) is powered by external power source (8′) connected to the base (28) suitable for receiving said device (5), which also has an eye protector (29) and a tip (30) conductive of UV light; the operation of the device (5) is made by means of buttons (31) strategically positioned in its structure (26).
